(19) United States
(12) Patent Application Publication
(10) Pub. No.: US 2025/0345592 A1
(43) Pub. Date: Nov. 13, 2025

(54) BIODEGRADABLE ION PATCH AND METHOD FOR MANUFACTURING THE SAME

(71) Applicant: SEOUL NATIONAL UNIVERSITY R&DB FOUNDATION, Seoul (KR)

(72) Inventors: Jieun Yun, Seongnam-si (KR); Kyung Eun Lee, Seongnam-si (KR); Jae Hwan Choi, Seongnam-si (KR); Seung Kyun Kang, Seoul (KR); Sung Geun Choi, Seongnam-si (KR)

(73) Assignee: SEOUL NATIONAL UNIVERSITY R&DB FOUNDATION, Seoul (KR)

(21) Appl. No.: 19/277,713

(22) Filed: Jul. 23, 2025

Related U.S. Application Data
(63) Continuation-in-part of application No. PCT/KR2024/011398, filed on Aug. 2, 2024.

(30) Foreign Application Priority Data
Aug. 4, 2023 (KR) 10-2023-0102393

(51) Int. Cl. A61N 1/04; A61K 9/00; A61K 9/70

(52) U.S. Cl. CPC A61N 1/0436; A61K 9/0009; A61K 9/7046; A61N 1/0496

(57) ABSTRACT

The present invention relates to a biodegradable iontophoretic patch and a method for manufacturing the same. The ion patch comprises a drug-loaded gel and a buffering gel, and by adding a pH adjuster to the buffering gel, it is possible to prevent excessive increases in pH during use of the ion patch. Therefore, the invention can be used in the manufacture of ion patches for enhancing skin absorption of cosmetic or pharmaceutical compositions.

### CROSS REFERENCE TO RELATED APPLICATIONS

This application is a Continuation in Part of International Application No. PCT/KR2024/011398 filed on Aug. 2, 2024, claiming priority based on Korean Patent Application No. 10-2023-0102393 filed on Aug. 4, 2023, the respective disclosures of which are incorporated herein by reference in their entirety.

### TECHNICAL FIELD

The present invention relates to a biodegradable iontophoretic patch and a method for manufacturing the same, and more particularly, to a technology for providing an ion patch made of a biodegradable material, the patch comprising a drug-loaded gel and a buffering gel, wherein a pH adjuster is added to the buffering gel to prevent excessive increase in pH value during use of the ion patch.

### BACKGROUND ART

Iontophoresis refers to a technique in which a microcurrent is applied to the skin to allow a charged target substance to penetrate into the skin by means of electrical repulsion. An iontophoretic patch is used to deliver a drug or a cosmetic substance, as a target substance, into the skin by utilizing electrical repulsion. When a patch loaded with the target substance is attached to the skin, a circuit is formed, allowing current to flow, and the target substance penetrates into the skin due to the electrical repulsion. It is known that the rate of penetration of such target substance is directly affected by the amount of current flowing through the skin.

Conventional iontophoretic devices generally attach two electrodes to the human skin, and each electrode is connected to an electrical device via wires. The target substance is placed on the electrode surface, and when the electrical device is activated, the target substance penetrates into the body. The electrical device is designed to control the amount and duration of current; however, a problem with such systems is that the patient must be connected to the electrical device via wires, which restricts the patient's daily movements and activities.

In addition, such systems are vulnerable to preservation of the mounted battery, have complicated assembly procedures resulting in low productivity and increased cost, and due to the multi-step reaction process, their efficacy is reduced, thereby limiting the ability to deliver cosmetic or pharmaceutical substances into the skin.

Recently, patch-type iontophoretic systems have been developed in the direction of integrating the electric circuit and power supply into a single patch. In such systems, the iontophoretic patch and battery are electrically connected to form a single patch. This form is regarded as an advancement compared to the previous system in which power was supplied externally through wires.

### DETAILED DESCRIPTION OF THE INVENTION

### Technical Problem

Accordingly, the inventors of the present invention have confirmed that, by loading a drug into a drug-loaded gel laminated on the cathode and including a pH adjuster in a buffering gel laminated on the anode, it is possible to both lower the pH value and buffer pH changes, thereby effectively suppressing excessive increase in pH when the ion patch is attached and used.

Therefore, an object of the present invention is to provide an ion patch comprising: a plate-shaped frame; an electrode layer comprising electrodes laminated on the frame; and a gel layer comprising a gel laminated on the electrodes.

Another object of the present invention is to provide a method for manufacturing an ion patch, the method comprising the steps of
forming an electrode layer by laminating electrodes on a plate-shaped frame; and
forming a gel layer by laminating a gel on the electrodes.

### Solution to the Technical Problem

The present invention relates to a biodegradable iontophoretic patch and a method for manufacturing the same. In the ion patch according to the present invention, a drug-loaded gel and a buffering gel are separately provided, and a pH adjuster is added to the buffering gel, thereby preventing an excessive increase in the pH value during use of the ion patch.

The present invention will be described in more detail below.

One aspect of the present invention is an ion patch comprising: a plate-shaped frame; an electrode layer comprising electrodes laminated on the frame; and a gel layer comprising a gel laminated on the electrodes.

Another aspect of the present invention is an ion patch comprising: an electrode layer including a pair of an anode and a cathode; and gel layers, respectively laminated on the anode and the cathode, performing different functions, wherein the gel laminated on the anode is a buffering gel including a pH adjuster, and the gel laminated on the cathode is a drug-loaded gel.

The ion patch may further comprise a frame located under the electrodes, and the frame may include a biodegradable polymer. The biodegradable polymer constituting the frame may include polybutylene adipate-co-terephthalate (PBAT).

The following means may be added to one or more of the above aspects to constitute respective embodiments.

In the present invention, the electrode layer may include a cathode and an anode disposed apart from the cathode.

In the present invention, the cathode and the anode may each independently have a thickness of 50 to 400 μm.

Preferably, the cathode may have a thickness of 50 to 350 μm, 50 to 300 μm, 100 to 400 μm, 100 to 350 μm, 100 to 300 μm, 150 to 400 μm, 150 to 350 μm, 150 to 300 μm, 200 to 400 μm, or 200 to 350 μm, and for example, may have a thickness of 200 to 300 μm, but is not limited thereto.

Also preferably, the anode may have a thickness of 50 to 350 μm, 50 to 300 μm, 50 to 200 μm, 50 to 150 μm, 100 to 400 μm, 100 to 350 μm, 100 to 300 μm, 100 to 250 μm, or 100 to 200 μm, and for example, may have a thickness of 100 to 150 μm, but is not limited thereto.

In the present invention, the gel laminated on the cathode and the gel laminated on the anode may be disposed apart from each other.

In the present invention, the gel layer laminated on the cathode and the gel layer laminated on the anode may each independently have a thickness of 0.4 to 2 mm, and preferably a thickness of 0.4 to 1.5 mm, 0.4 to 1.2 mm, 0.4 to 1 mm, 0.8 to 2 mm, 0.8 to 1.5 mm, or 0.8 to 1.2 mm, and for example, may have a thickness of 0.8 to 1 mm, but is not limited thereto.

In the present invention, the cathode may include a biodegradable material capable of a skin-friendly reduction reaction, and may include one or more selected from a transition metal oxide, a metal, and a biodegradable polymer. The cathode may be made of molybdenum trioxide (MoO₃), but is not limited thereto.

Since molybdenum trioxide undergoes insertion of Na⁺ ions or the like into its lattice structure during the electrochemical reaction process, it does not consume H⁺ or OH⁻ ions, and thus side reactions such as pH change can be suppressed. Therefore, such reactions can be defined as skin-friendly reduction reactions. In the present invention, not only molybdenum trioxide but also any material in which such side reactions are suppressed may be included in the cathode material.

In the present invention, the cathode may include a biodegradable polymer and a crosslinking agent.

Since the biodegradable polymer is hydrophilic, water can be well adsorbed into the polymer. Conventionally, when such a biodegradable polymer is used, swelling occurs as water, serving as an electrolyte, permeates into the polymer during operation of the ion patch, which results in the polymer failing to retain the cathode material, leading to detachment. Due to this, electron transfer from the cathode material becomes difficult, and normal electrochemical reactions cannot occur, resulting in performance degradation.

In contrast, in the present embodiment, a crosslinking agent is added to the polymer to suppress the movement and swelling of the biodegradable polymer, while allowing the biodegradable polymer to securely retain the cathode material during operation, thereby maintaining performance.

The cathode may include a coating material based on a biodegradable conductive substance on the contact surface with the frame. The coating material may include a coating of molybdenum (Mo) or titanium (Ti), but is not limited thereto.

The coating material may have a thickness of 5 to 30 μm, and preferably a thickness of 5 to 25 μm, 5 to 20 μm, 5 to 15 μm, 10 to 30 μm, 10 to 25 μm, or 10 to 20 μm, and for example, may have a thickness of 10 to 15 μm, but is not limited thereto.

In the present invention, the gel laminated on the cathode may be a drug-loaded gel including a loaded drug.

The loaded drug may be at least one selected from the group consisting of caffeine, niacinamide, and adenosine, but is not limited thereto.

In the present invention, the anode may include a biodegradable metal. The anode may be made of magnesium (Mg), but is not limited thereto.

In the present invention, the gel laminated on the anode may be a buffering gel including a pH adjuster, and the pH adjuster may be citric acid.

The citric acid may be included in the buffering gel at a concentration of 150 to 500 mM, and preferably at a concentration of 150 to 400 mM, 150 to 300 mM, 150 to 250 mM, 200 to 500 mM, 200 to 400 mM, 200 to 300 mM, 200 to 250 mM, 250 to 500 mM, or 250 to 400 mM, and for example, may be included at a concentration of 250 to 300 mM, but is not limited thereto.

In the present invention, the electrode layer may further include a current regulator connecting the cathode and the anode.

The current regulator may be made of a mixture of biodegradable metal particles and a biodegradable polymer. The current regulator may include molybdenum (Mo) or titanium (Ti), but is not limited thereto. The current regulator may serve as a resistor based on a mixture in which biodegradable metal particles and a biodegradable polymer are mixed at a certain ratio. The current regulator may allow for the adjustment of conductivity by controlling, for example, the volume ratio of internal particles.

The current regulator may be formed by mixing polybutylene adipate-co-terephthalate (PBAT), but is not limited thereto.

In the present invention, the concentration of metal particles in the current regulator may be 10 to 40 vol %.

In addition, in the present invention, the gel layer may be composed of two or more layers each independently comprising at least two types selected from the group consisting of alginate, gelatin, agar, maltose, and glycerol, and, for example, may be composed of two layers respectively comprising gelatin and agar independently, but is not limited thereto. The functions of each layer will be described in more detail below.

In the present invention, the drug-loaded gel may include at least one selected from the group of biodegradable ionic polymers such as alginate, carboxymethyl cellulose (CMC), and dextran sulfate (DS).

The biodegradable ionic polymer used in the drug-loaded gel may include functional groups bearing ions such as COO⁻ and SO₃²⁻. When a drug carrying a counterion (+) is loaded into such an ionic polymer, only the movement of the positively charged drug and the positively charged ion previously bound to the ionic functional group of the polymer is possible during the operation of the ion patch, thereby enabling ion movement in a single direction.

Since ions move with adsorbed water, controlling the direction of ion movement also controls the flow of water in a single direction; this phenomenon is called electroosmosis. In the present invention, the water flow generated by such electroosmosis can carry the dissolved drug along with it, thereby enabling the delivery of a significantly greater amount of drug using the same electric power.

In the present invention, the buffering gel may include at least one selected from the group consisting of agarose, maltose, and glycerol, each independently. It is preferable that the polymer used in the buffering gel is a biodegradable polymer with good ionic conductivity.

In the present invention, the concentration of the gel may be 2 to 10 wt %, and preferably 2 to 8 wt %, 2 to 6 wt %, 2 to 4 wt %, 4 to 10 wt %, or 4 to 8 wt %, and for example, may be 4 to 6 wt %, but is not limited thereto.

In the present invention, one or more of the drug-loaded gel layer and the buffering gel layer may further include a skin-adhesive gel layer, and the skin-adhesive gel layer may include gelatin and an ion-conductive biodegradable polymer.

Since gels such as agar or alginate have no adhesiveness, they may easily detach upon attachment to the skin, resulting in poor operational stability. Therefore, by laminating a gelatin-based gel layer, which has good adhesion to the skin, at the portion contacting the skin, skin adhesion can be improved. The appropriate concentration of gelatin in the gel layer may be from 1 wt % to 4 wt %.

However, if only gelatin is used as the gel layer, problems may arise due to the low ion conductivity of gelatin-based gels. This can reduce the current of the ion patch and thereby decrease the drug delivery rate. Accordingly, in the present embodiment, to ensure both a high drug delivery rate and sufficient adhesion, a laminated structure is adopted in which a skin-adhesive gel layer is positioned on the side of the gel layer that contacts the skin.

Another aspect of the present invention is a method for manufacturing an ion patch, the method comprising:
a step of forming an electrode layer by laminating electrodes on a plate-shaped frame; and
a step of forming a gel layer by laminating a gel on the electrodes.

In the present invention, the frame may be PLGA or PGLA.

In the present invention, the step of forming the electrode layer may include forming a cathode and an anode disposed apart from the cathode.

The cathode may be made of molybdenum trioxide (MoO₃), but is not limited thereto.

The cathode may further include a coating material of molybdenum (Mo) or titanium (Ti) on the contact surface with the frame, but is not limited thereto.

The anode may be made of magnesium (Mg), but is not limited thereto.

In the present invention, the step of forming the gel layer may include disposing the gel laminated on the cathode and the gel laminated on the anode so as to be spaced apart from each other.

The gel laminated on the cathode may be a drug-loaded gel including a loaded drug.

The gel laminated on the anode may be a buffering gel including a pH adjuster, and the pH adjuster may be citric acid.

In the present invention, the manufacturing method may further include a step of forming a current regulator connecting the cathode and the anode.

The current regulator may be made of molybdenum (Mo) or titanium (Ti), but is not limited thereto.

The current regulator may be coated with PBAT, but is not limited thereto.

In the present invention, the step of forming the gel layer may be performed by laminating at least one selected from the group consisting of alginate, gelatin, agar, maltose, and glycerol, and, for example, by laminating alginate, but is not limited thereto.

In the present invention, the step of forming the gel layer may be performed by laminating two or more layers each independently comprising two or more types selected from the group consisting of alginate, gelatin, agar, maltose, and glycerol, and, for example, by laminating two layers respectively comprising gelatin and agar independently, but is not limited thereto.

### Effects of the Invention

The present invention relates to a biodegradable iontophoretic patch and a method for manufacturing the same. The ion patch comprises a drug-loaded gel and a buffering gel, and by adding a pH adjuster to the buffering gel, it is possible to prevent excessive increase in pH value during use of the ion patch. Therefore, it can be used in the manufacture of ion patches for enhancing skin absorption of cosmetic compositions or pharmaceutical compositions.

### BRIEF DESCRIPTION OF THE DRAWINGS

FIG. 1a is a schematic view of an ion patch manufactured according to one embodiment of the present invention.

FIG. 1b is a graph showing current signals measured from ion patches manufactured with varying thicknesses of MoO₃ according to one embodiment of the present invention.

FIG. 2a is a schematic view and a photograph of an ion patch manufactured with the thickness of MoO₃ paste selected as 300 μm according to one embodiment of the present invention.

FIG. 2b is a graph showing current signals measured from an ion patch manufactured with the thickness of MoO₃ paste selected as 300 μm according to one embodiment of the present invention.

FIG. 2c is a discharge graph measured from ion patches manufactured with or without the addition of citric acid, a crosslinking agent, to a MoO₃ and PAAc (polyacrylic acid) polymer paste according to one embodiment of the present invention.

FIG. 2d is an image confirming whether diffusion of cathode material occurred due to polymer swelling after discharging for 1 hour at 300 μA, in ion patches manufactured with or without the addition of citric acid, a crosslinking agent, to a MoO₃ and PAAc polymer paste according to one embodiment of the present invention.

FIG. 3a is a schematic view of an ion patch in which the cathode is coated with molybdenum (Mo) according to one embodiment of the present invention.

FIG. 3b is a graph showing pH value changes under conditions of pH 7.4, as measured from an ion patch with a molybdenum-coated cathode according to one embodiment of the present invention.

FIG. 3c is a graph showing pH value changes under conditions of pH 5.5, as measured from an ion patch with a molybdenum-coated cathode according to one embodiment of the present invention.

FIG. 4a is an illustration showing the pH values of the skin surface according to different regions of the human body.

FIG. 4b is a graph showing pH value changes under conditions of pH 4.2, as measured from an ion patch according to one embodiment of the present invention.

FIG. 5a is a schematic view of an ion patch for confirming pH control depending on the presence or absence of citric acid, according to one embodiment of the present invention.

FIG. 5b is a schematic view of an ion patch showing the measurement area for confirming pH control depending on the presence or absence of citric acid, according to one embodiment of the present invention.

FIG. 6a is a schematic view of an ion patch and a skin model for confirming the drug absorption rate into the skin depending on current density, according to one embodiment of the present invention.

FIG. 6b is a graph showing the drug absorption rate into the skin from the ion patch depending on current density, according to one embodiment of the present invention.

FIG. 7a is a schematic view of an electrode structure and a skin model for confirming the drug absorption rate into the skin from the ion patch depending on whether iontophoresis is applied, according to one embodiment of the present invention.

FIG. 7b is a graph showing the drug absorption rate into the skin from the ion patch depending on whether iontophoresis is applied, according to one embodiment of the present invention.

FIG. 8a is a graph showing the absorption rate of niacinamide into the skin from the ion patch depending on whether iontophoresis is applied, according to one embodiment of the present invention.

FIG. 8b is a graph showing the absorption rate of adenosine into the skin from the ion patch depending on whether iontophoresis is applied, according to one embodiment of the present invention.

FIG. 9a is a schematic view of single-gel and double-gel ion patch models according to one embodiment of the present invention.

FIG. 9b is a graph showing current intensity in single-gel and double-gel ion patch models according to one embodiment of the present invention.

FIG. 10a is a graph showing current intensity in a gelatin gel-applied ion patch model according to one embodiment of the present invention.

FIG. 10b is a graph showing current intensity in an alginate gel-applied ion patch model according to one embodiment of the present invention.

FIG. 11a is a schematic view of ion patch models with different types of gels applied, according to one embodiment of the present invention.

FIG. 11b is a graph comparing the ion absorption amounts of gelatin- and alginate-applied ion patch models, according to one embodiment of the present invention.

FIG. 12a is a schematic view of an ion patch model and a skin model for measuring ion absorption amounts at different skin depths, according to one embodiment of the present invention.

FIG. 12b is a graph showing ion absorption amounts of a drug-loaded gel, according to one embodiment of the present invention.

FIG. 13 is a schematic view of an ion patch manufactured according to one embodiment of the present invention.

### MODE FOR CARRYING OUT THE INVENTION

Hereinafter, the present invention will be described in more detail with reference to the following examples. However, these examples are provided only for illustrative purposes, and the scope of the present invention is not limited thereto.

Throughout this specification, unless otherwise stated, “%” used to indicate the concentration of a specific substance means (weight/weight) % for solid/solid, (weight/volume) % for solid/liquid, and (volume/volume) % for liquid/liquid.

### Example 1: Configuration of Ion Patch

To determine the appropriate thickness for using molybdenum trioxide (MoO₃) as the electrode in the configuration of the ion patch, electrode structures were fabricated with MoO₃ applied at thicknesses of 150, 200, and 400 μm, respectively, and the current signal was measured using an ammeter.

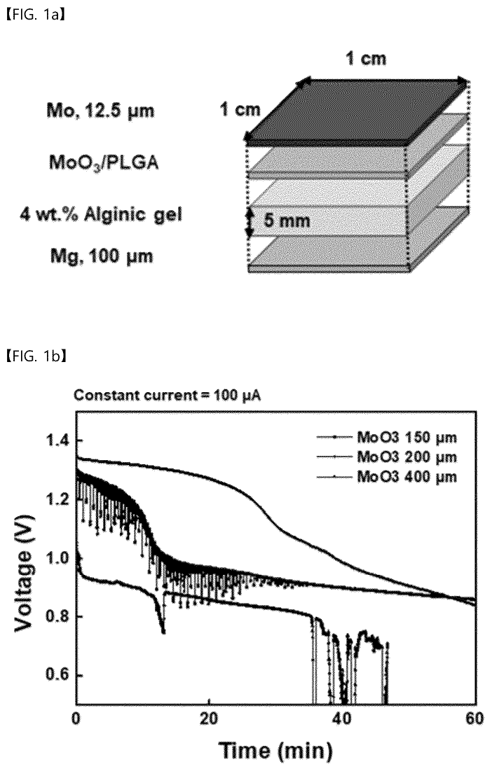

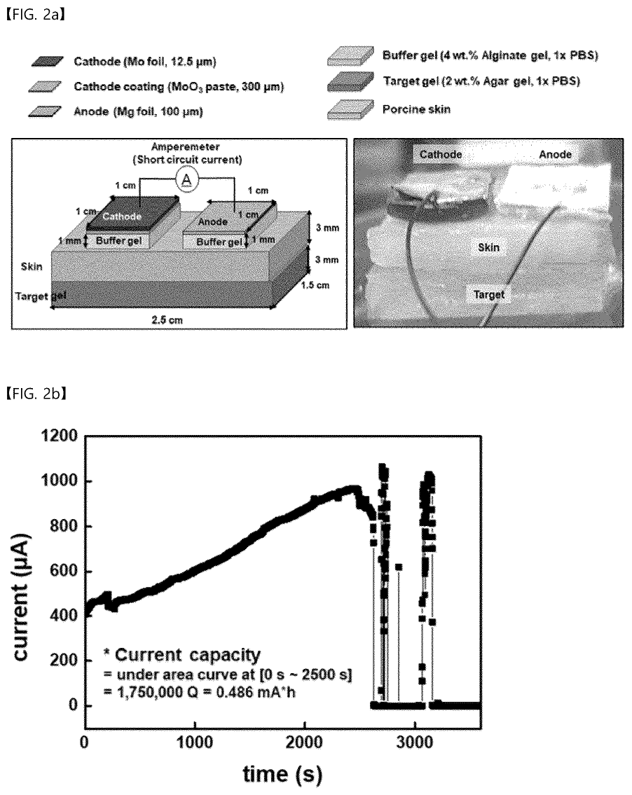

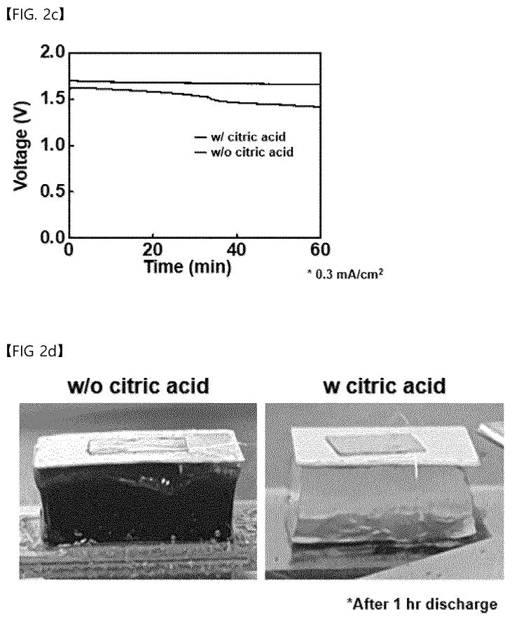

As shown in FIGS. 1a and 1b, as the thickness of MoO₃ increases, the current capacity increases and ion diffusion decreases; thus, a thickness of 200 μm was determined to be the most optimal. Subsequently, an ion patch model capable of implementing iontophoretic operation was fabricated using MoO₃ with a selected thickness of 300 μm as the cathode, as shown in FIG. 2a, and the current signal was measured.

As shown in FIG. 2b, the current capacity measured from the ion patch operation model was 0.486 mAh, which is higher than the required iontophoresis threshold of 0.15 mAh, indicating that the model is capable of generating microcurrents within an appropriate range.

### Example 2: Ensuring Operational Stability Using a Crosslinking Agent

In water-electrolyte-based galvanic ion patches, during operation, the cathode composed of electrode material and a polymer binder absorbs moisture due to the hydrophilicity of the polymer, causing swelling and resulting in reduced operational stability. To partially address this issue, a method utilizing a crosslinking agent can be employed.

As shown in FIG. 2c, a comparison based on the presence or absence of the crosslinking agent citric acid reveals that when no crosslinking agent is used, the polymer binder swells over time, causing the electrode material to detach and spread, leading to unstable electrochemical reactions at the cathode and a resulting decrease in operating voltage. In contrast, when the crosslinking agent is used, the electrode material remains stably fixed within the polymer binder over time, maintaining a stable electrochemical reaction and thereby sustaining the operating voltage.

As shown in FIG. 2d, when the crosslinking agent is not used, the electrode material diffuses into the electrolyte after operation. In contrast, when the crosslinking agent is used, the electrode material remains fixed even after operation.

### Example 3: Conditioning pH Value in the Ion Patch

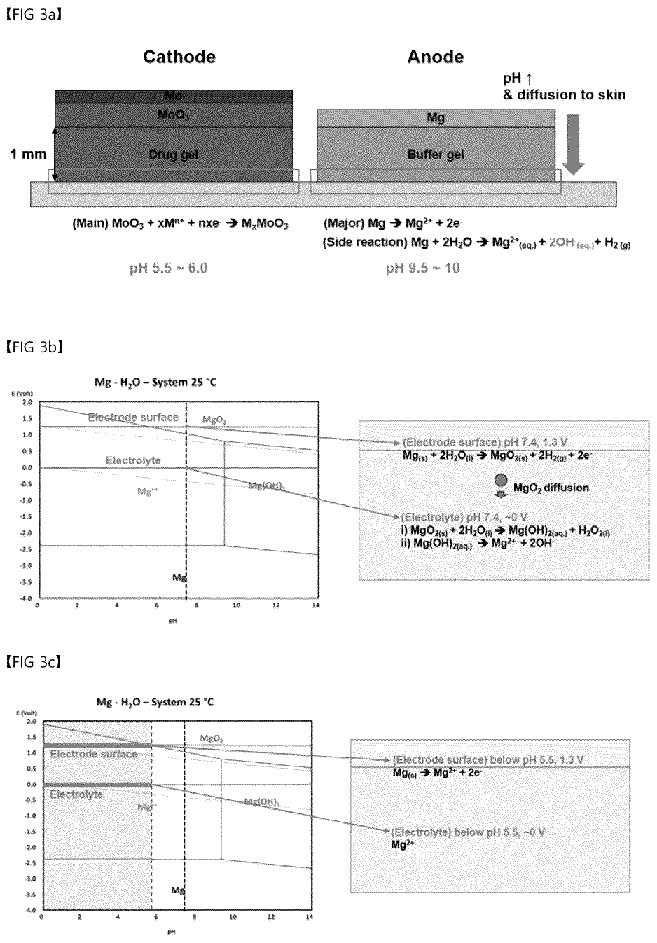

During operation, the ion patch may cause an increase in pH at the anode. To partially address this issue, a method of coating the MoO₃ cathode with molybdenum (Mo), as shown in FIG. 3a, can be employed.

As shown in FIG. 3b, when the pH is 7.4, MgO₂ increases on the anode surface, resulting in the generation of OH⁻ ions in the electrolyte, which in turn increases the pH value. From this result, it can be inferred that suppressing the formation of MgO₂ on the electrode surface may be a way to prevent an increase in pH.

As shown in FIG. 3c, when the pH is lowered to 5.5, the formation of MgO₂ on the anode surface is suppressed, preventing the generation of OH⁻ ions in the electrolyte, and thus the pH value did not increase.

### Example 3: Exploration of Optimal pH Conditions

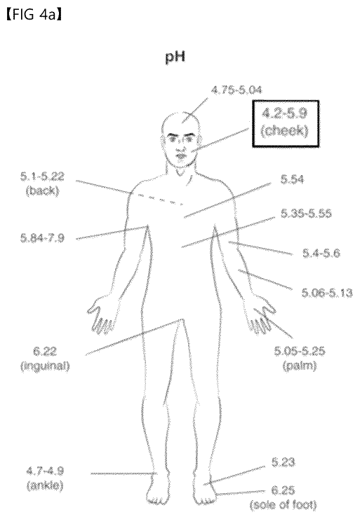

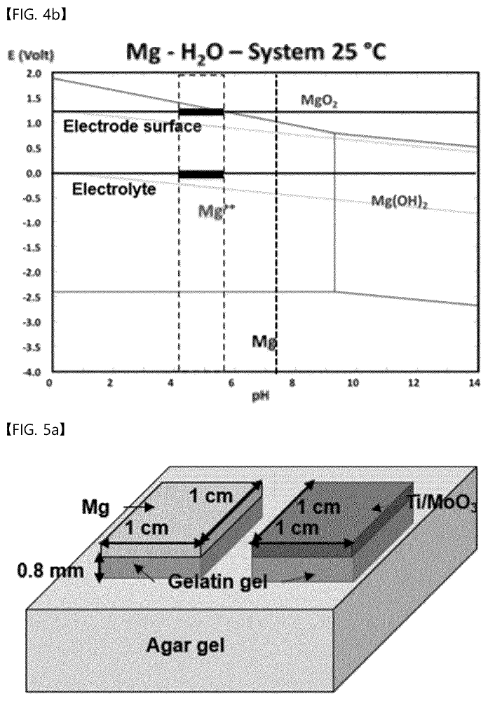

As shown in FIG. 4a, based on biocompatibility with reference to the facial area where the ion patch is applied, a pH value of 4.2 is appropriate; accordingly, the application conditions of the electrode structure were adjusted to this pH.

As shown in FIG. 4b, the results of applying the electrode structure under pH 4.2 conditions showed that the concentration of Mg²⁺ ions remained stable, similar to the results under pH 5.5 conditions.

To control the pH value, 250 mM of citric acid was added to the gel laminated on the anode, and a control group without citric acid was prepared for comparison.

TABLE 1

| Gelatin | | | | Citric | Citric Acid | |
|---|---|---|---|---|---|---|
| Gel | PBS | Glycerol | Syrup | Acid | Concentration | pH |
| Citric Acid | 8 ml | 6.3 ml | 6.5 ml | 1 g | 250 mM | 3 |
| Control | 8 ml | 6.3 ml | 6.5 ml | 0 g | 0 mM | 7.4 |

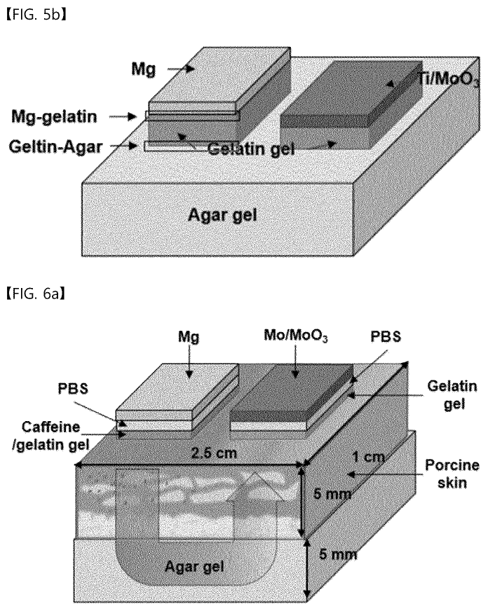

The conditions of the electrode structures shown in FIGS. 5a and 5b are as follows:
Current: 100 μA
Duration: 1 hour
Volume of gelatin gel: 0.08 cm³
Measured amount of OH⁻ generated on the Mg surface: 3.73 mol

### Measured Citric Acid Content in the Gelatin Gel:

( [symbol] : 0 mol; Article condition: 20 mol; Harsh condition: 40 mol)

TABLE 2

| Condition | pH @ Mg - Gelatin | pH @ Gelatin - Agar |
|---|---|---|
| Citric Acid Added | 3 | 3 |
| Control (No Citric Acid) | 9.5~10 | 8 |

As shown in Table 2, analysis of the model in which the operation of the ion patch was implemented revealed that, in the ion patch containing a gel with added citric acid, the pH value was consistently measured as 3 regardless of the measurement site. However, in the case without citric acid, the pH value varied by measurement site, being measured as 8 or as high as 9.5 to 10.

From this, it can be concluded that the addition of citric acid effectively suppresses the increase in pH value.

### Example 4: Optimization of Current Density to Maximize Drug Absorption Rate

To enhance the absorption efficiency of the drug loaded in the provided gel, the current density applied to the ion patch was adjusted. Specifically, caffeine was added to the gelatin gel laminated on the anode of the ion patch, and various current densities of 50, 100, and 150 μA were applied. The amount of caffeine absorbed into the skin was then measured and compared.

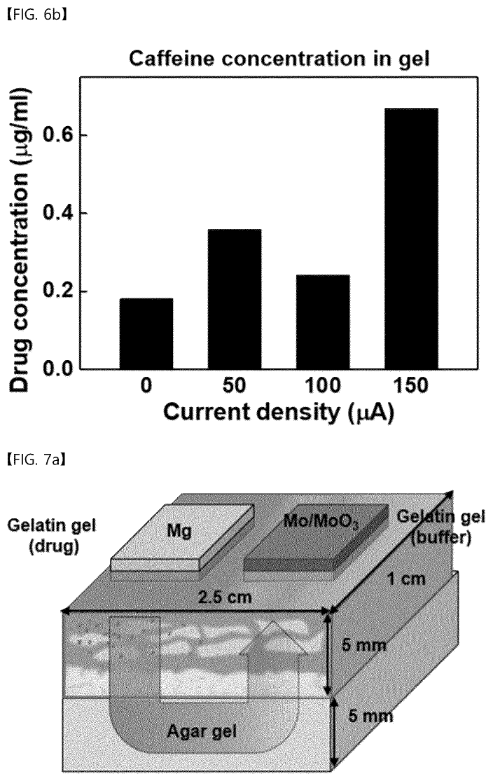

As shown in FIGS. 6a and 6b, the amount of caffeine absorbed was highest in the order of 150, 50, and 100 μA current densities. In particular, the highest absorption rate was observed at 150 μA, suggesting that this current density is the most suitable for application in the ion patch.

### Example 5: Comparison of Drug Absorption Rate Depending on Application of Iontophoresis

To compare the difference in drug absorption rate depending on whether iontophoresis is applied, the amount of drug absorbed into human skin and reaching internal tissue was measured at 5 mm and 10 mm depths from the skin surface after applying the ion patch.

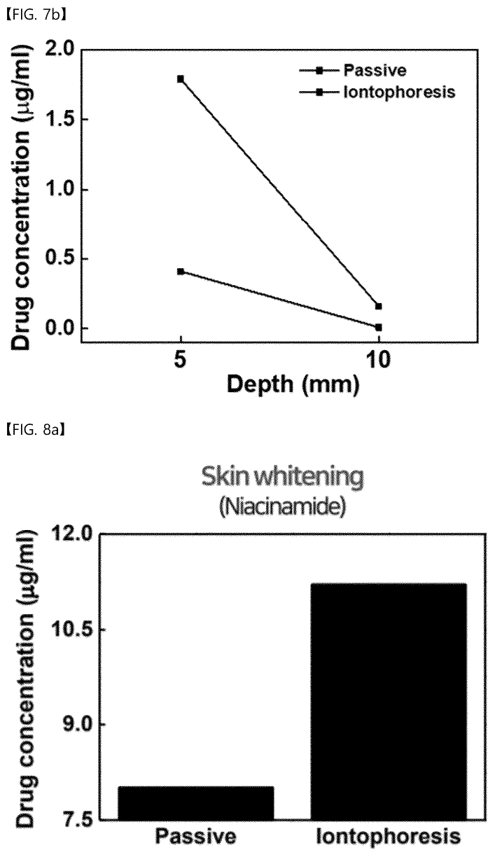

As shown in FIGS. 7a and 7b, even at the 5 mm depth, the ion patch showed significantly higher drug absorption compared to the control. Notably, at the 10 mm depth, almost no drug absorption was detected from the control group, while a measurable amount of drug was absorbed from the ion patch. From these results, it was confirmed that the application of iontophoresis enhances not only the absorption rate but also the penetration depth of the drug into the skin.

Subsequently, the skin absorption of active ingredients was examined using a skin-whitening ion patch comprising a gel containing niacinamide and an anti-aging ion patch comprising a gel containing adenosine.

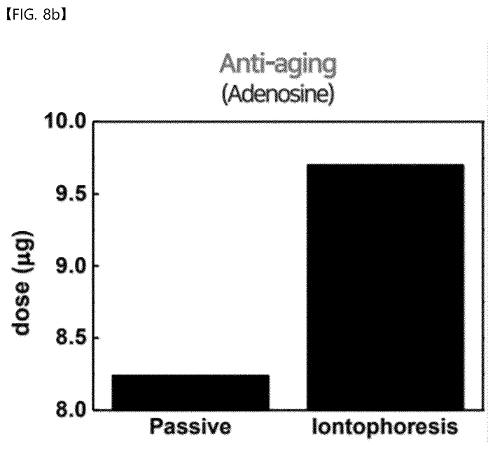

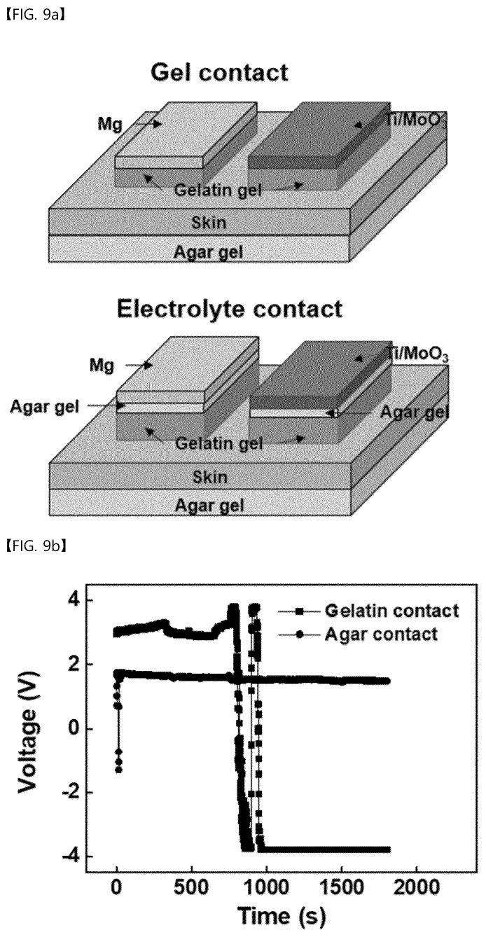

As shown in FIGS. 8a and 8b, the application of iontophoresis resulted in a significantly higher absorption amount of the active ingredients compared to the control group.

### Example 7: Optimization of Gel Structure and Composition

### 7-1. Ion Absorption Control Via Dual-Gel Structure

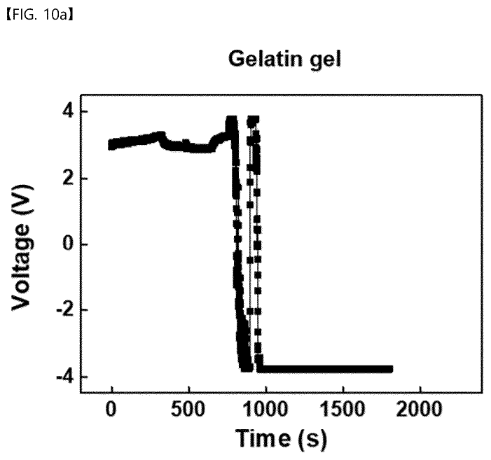

In constructing the ion patch operation model, two configurations were prepared as shown in FIG. 9a: one using a gelatin gel alone, and the other using a dual-gel structure by adding an agar gel to the gelatin gel.

As shown in FIG. 9b, the dual-gel model with added agar showed slower ion absorption and reduced current intensity. Therefore, this configuration allows for the adjustment of drug absorption according to specific needs.

### 7-2. Ion Absorption Control Depending on Gel Material

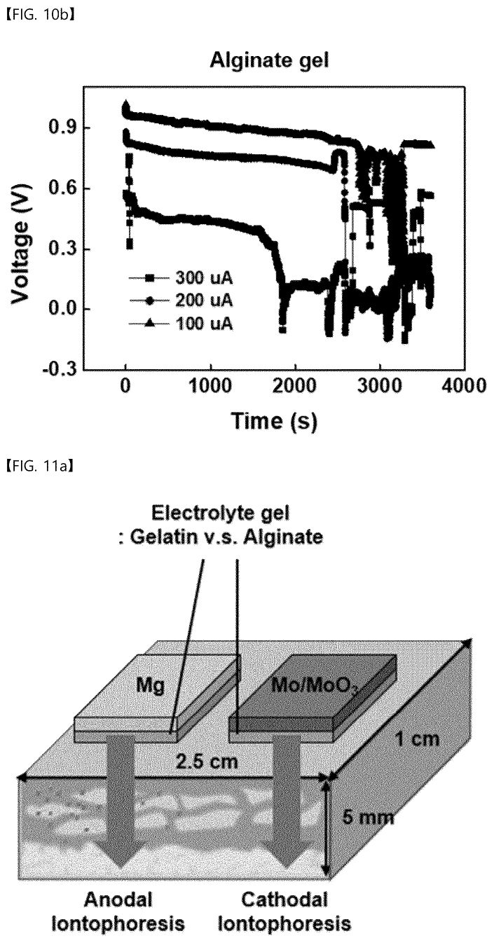

The effect of replacing the gelatin gel with alginate in the electrode structure was also compared. As shown in FIGS. 10a and 10b, compared to the use of gelatin gel, alginate gel resulted in increased ion diffusion and higher current intensity, confirming that the type of gel material significantly affects ion transport behavior.

### 7-3. Ion Absorption Control Depending on Electrode Selection

An analysis was conducted to determine how the absorption of active ingredients into the skin differs depending on which electrode—cathode or anode—the drug-loaded gel is laminated onto.

Specifically, electrolyte gels composed of gelatin or alginate were applied to either the cathode or anode, and the amount of absorbed ions was measured.

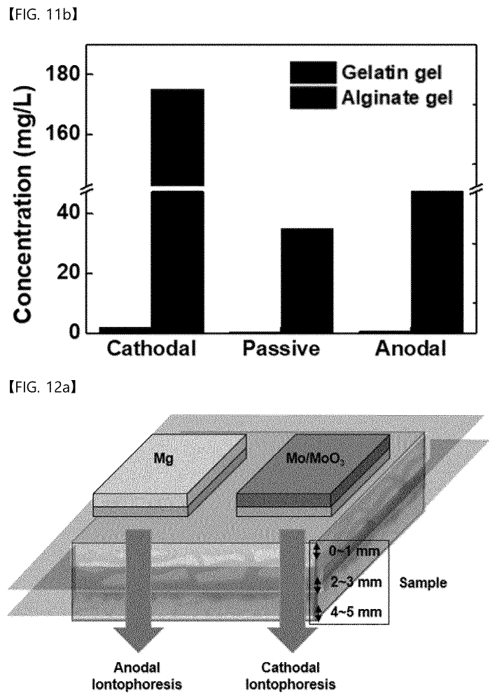

As shown in FIGS. 11a and 11b, the alginate gel maintained a stable current of 200 μA for two hours. Particularly, when used on the cathode, it showed a much higher ion absorption amount compared not only to the passive control but also to the case when applied on the anode. On the other hand, the gelatin gel, which was limited to 50 μA for only one hour, showed negligible ion absorption compared to alginate.

This result suggests that the superior diffusivity of alginate enhances iontophoretic delivery efficiency.

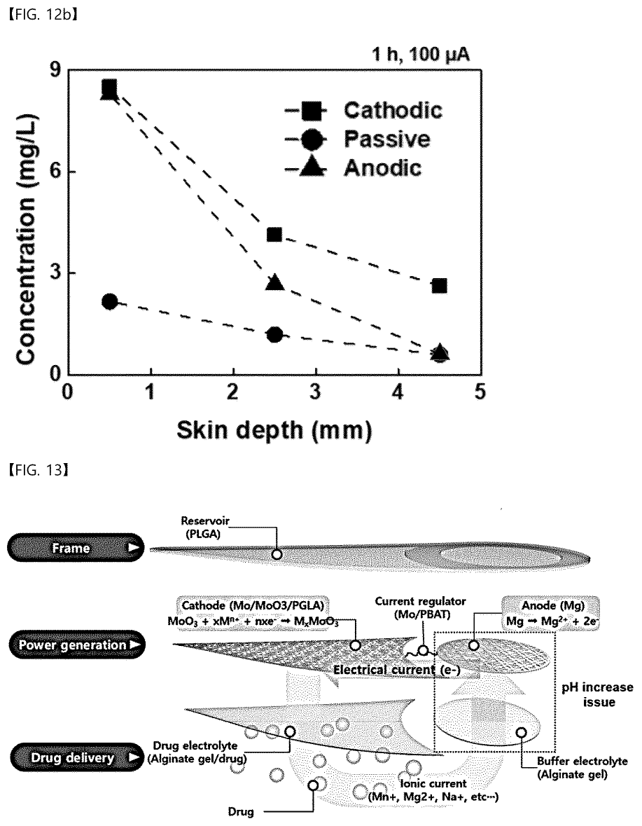

Next, as shown in FIG. 12a, drug penetration into skin at different depths (0-1 mm, 2-3 mm, and 4-5 mm) was measured and compared according to the type of electrode laminated with the drug-loaded gel.

As shown in FIG. 12b, the overall amount of drug absorbed from the gel into the skin surface was similar for both the cathode and the anode. However, at a depth of 5 mm, only the ions delivered from the gel laminated on the cathode were detected.

As a result, a biodegradable ion patch as shown in FIG. 13 was developed.

1. An ion patch comprising:
an electrode layer including a pair of an anode and a cathode; and
gel layers respectively laminated on the anode and the cathode, performing different functions, wherein the gel laminated on the anode is a buffering gel including a pH adjuster, and
the gel laminated on the cathode is a drug-loaded gel.

2. The ion patch of claim 1,
wherein the ion patch further comprises a frame located beneath the electrodes, and
the frame comprises a biodegradable polymer including polybutylene adipate-co-terephthalate (PBAT).

3. The ion patch of claim 1,
wherein the cathode and the anode each independently have a thickness of 50 to 400 μm.

4. The ion patch of claim 1,
wherein the gel layer laminated on the cathode and the gel layer laminated on the anode each independently have a thickness of 0.4 to 2 mm.

5. The ion patch of claim 1,
wherein the cathode comprises a biodegradable material capable of a skin-friendly reduction reaction, and includes one or more selected from transition metal oxides, biodegradable metals, and biodegradable polymers.

6. The ion patch of claim 1,
wherein the cathode comprises a biodegradable polymer and a crosslinking agent.

7. The ion patch of claim 2,
wherein the cathode further comprises a coating layer based on a biodegradable conductive material on a contact surface with the frame.

8. The ion patch of claim 1,
wherein the anode comprises a biodegradable metal.

9. The ion patch of claim 1,
wherein the electrode layer further comprises a current regulator connecting the cathode and the anode.

10. The ion patch of claim 9,
wherein the current regulator comprises a mixture of biodegradable metal particles and a biodegradable polymer.

11. The ion patch of claim 1,
wherein the drug-loaded gel comprises at least one selected from the group of biodegradable ionic polymers including alginate, carboxymethyl cellulose (CMC), and dextran sulfate (DS).

12. The ion patch of claim 1,
wherein the buffering gel independently comprises at least one selected from the group consisting of agarose, maltose, and glycerol.

13. The ion patch of claim 1,
wherein one or more of the drug-loaded gel layer and the buffering gel layer further comprises a skin-adhesive gel layer,
and the skin-adhesive gel layer comprises gelatin and an ion-conductive biodegradable polymer.
